(19) United States
(12) Patent Application Publication
(10) Pub. No.: US 2007/0173934 A1
(43) Pub. Date: Jul. 26, 2007

(54) Devices to protect features on an implant and methods of use

(75) Inventors: Charles Anthony Dickinson, Millington, TN (US); Hai H. Trieu, Cordova, TN (US); Douglas Neil Baker, Collierville, TN (US); Marco D. Capote, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/336,310

(22) Filed: Jan. 20, 2006

(51) Int. Cl. A61F 2/02

(52) U.S. Cl. 623/11.11; 623/17.11; 623/901; 606/61

(57) ABSTRACT

Protectors and methods of mounting protectors to features on implants within patients. The protectors attach to the features and provide protection to the patient by preventing or reducing injury that may be caused by the feature. The protectors may also be removable such that the feature can be exposed during future revision procedures. A variety of attachment methods may also be used to attach the protector to the feature. Insertion tools may be used for attaching the protectors to the features.

### BACKGROUND

The present application is directed to devices and methods to protect features on an implant and, more specifically, to a protective devices that attach to a feature on an implant and methods and devices for attaching the protective device to the feature.

Implants are positioned within the body in a wide variety of medical applications. Examples of implants may include a vertebral rod that extends along the spine, a vertebral plate that attaches to one or more vertebral members, and an intervertebral implant that is positioned between vertebral members. Implants are also used in a variety of non-vertebral applications. These implants may include one or more features that may be protected or otherwise isolated after installation of the implant. The features may be part of the implant itself such as an end of the vertebral rod, or a related element that is associated with the implant, such as a fastener that attaches the implant within the patient.

One reason to protect these features is to prevent injury to the patient. After the implant is installed within the body, these features may cause tissue irritation, tissue infection, and unintentional trauma to adjacent tissues and organs. These injuries may cause discomfort to the patient, and may elevate to an extent that additional surgical procedures are necessary to correct the problem.

Another reason to protect the implant features is to facilitate additional, later-occurring medical procedures. In these applications, the implant is initially introduced to the patient in a first procedure, and then revised in a subsequent procedure. The subsequent procedure may be required for various reasons such as adjustment, removal, or utilization of the feature. The amount of time between the procedures may result in tissue ingrowth or attachment that would prevent or limit the ability to access the feature in the later procedure.

### SUMMARY

The present application is directed to protectors and methods of mounting protectors to features on implants within patients. The protectors attach to the features and provide protection to the patient by preventing or reducing injuries that may be caused by the feature. The protectors may also be removable such that the feature can be exposed during future revision procedures. A variety of attachment methods may also be used to attach the protector to the feature. Insertion tools may be used for attaching the protectors to the features.

### BRIEF DESCRIPTION OF THE DRAWINGS

FIG. 1 is a partial perspective view of a protector and a feature according to one embodiment.

FIG. 2 is a partial cross section view of a protector attached to a feature according to one embodiment.

FIG. 3 is a perspective view of protectors attached to features of an implant according to one embodiment.

FIG. 4 is a schematic view of a protector according to one embodiment.

FIG. 5 is a partial perspective view of a protector and a feature according to one embodiment.

FIG. 6 is an isometric view of a protector extending over a feature according to one embodiment.

FIG. 7 is a cross section view of a protector extending over a feature according to one embodiment.

FIG. 8 is a cut away view of a protector attached to a feature according to one embodiment.

FIG. 9 is a perspective view of a protector according to one embodiment.

FIG. 10 is a perspective view of an insertion tool according to one embodiment.

FIG. 11 is a partial perspective view of an insertion tool and protector according to one embodiment.

FIGS. 12A-12D are schematic views of a method of attachment of a protector onto a feature according to one embodiment.

### DETAILED DESCRIPTION

The present application is directed to protectors that are attached to features that have been implanted within patients, and methods of attachment. The protectors attach to the features and provide protection to the patient by preventing or reducing injury that may be caused by the feature. The protectors may also be removable such that the feature can be exposed during a future revision procedure. A variety of attachment methods may also be used to attach the protector to the feature, and include a variety of insertion tools.

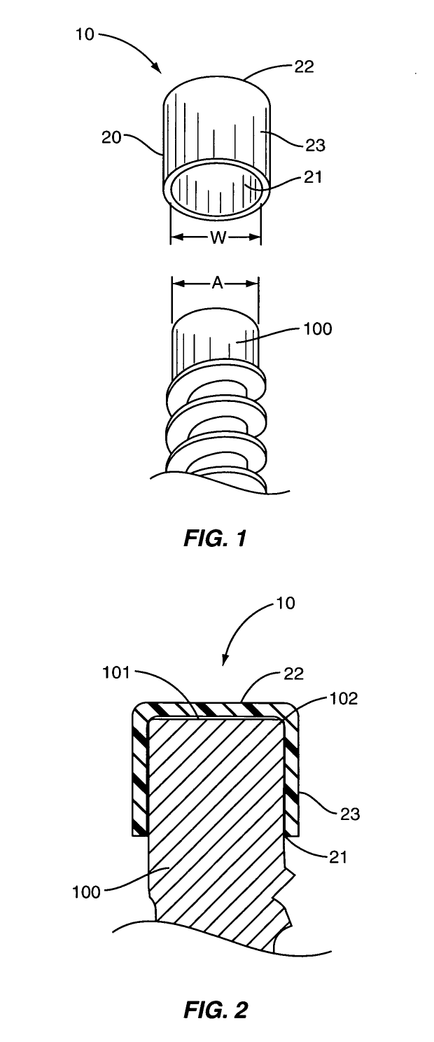

FIG. 1 illustrates one embodiment of a protector 10 that attaches to a feature 100. The protector 10 includes a body 20 having an end 22 and sidewall 23 that lead to an opening 21. Protector 10 attaches to the feature 100 to shield the patient from possible injury caused by the exposed feature 100. In one embodiment, the protector 10 is a thin member. In one embodiment, the protector is deformable.

In one embodiment, the protector 10 is constructed of an elastic material that can be stretched to attach to the feature 100. In the embodiment of FIG. 1, opening 21 includes a width W prior to attachment that is smaller than a width A of the feature 100. The elastic material that forms the protector 10 can be enlarged to increase the size of the opening 21 to fit over the feature 100. The enlarged body 20 fits over the feature 100 with sidewalls 23 extending along the lateral sides and the end 22 covering a top edge 101 of the feature 100. After placement over the feature 100, the protector 10 is reduced towards the starting size. This causes the protector 10 to be placed in tension and remain attached to the feature 100. FIG. 2 illustrates the protector 10 attached to the feature 100. The protector 10 includes a thickness to shield the feature 100. By way of example, protector 10 would shield the patient from a potentially sharp top edge 101 or corner 102. In the embodiment of FIG. 2, protector 10 conforms tightly to the feature 100.

The amount of force applied in an elastic body embodiment may vary depending upon the amount of enlargement, and the material. Examples of elastic materials may include silicone and polyurethane. In one embodiment, the elastic material is an elastomer that stretches during installation. Examples of elastomers include silicone-polyurethane copolymers, and polyolefin rubbers. The protector 10 may also be formed by other materials, and have different attachment methods. In one embodiment, the protector 10 is a deformable plastic that deforms during installation. Examples of deformable plastics include polyethylene, polypropylene, and polyester. In one embodiment, the protector 10 is constructed from a rigid plastic that may screw on, snap on, or be press fit. Examples of rigid plastics include polyetheretherketone, polysulfone, and polyimide.

In one embodiment as illustrated in FIG. 2, the body 20 conforms to make contact along the entirety of the feature 100. The sidewalls 23 are positioned against the lateral edges, and the end 22 is positioned against the top edge 101 of the feature 100. In other embodiments, body 20 makes more limited contact against the feature 100.

The term “feature.” is used generally herein to refer to an implant that is positioned within patient. The feature 100 may be the entirety of the implant, or a section of the implant. The feature may further comprise a fastener that attaches an implant within the patient. An implant may include a single feature 100, or multiple features 100.

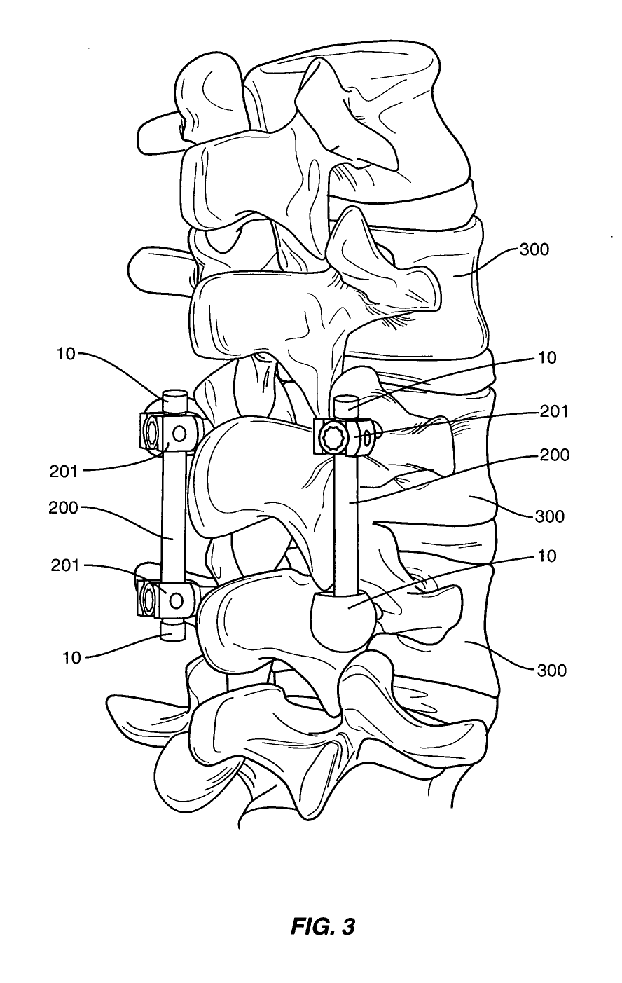

FIG. 3 illustrates one embodiment of an implant having multiple features 100. FIG. 3 specifically illustrates a pair of vertebral rods 200 connected by fasteners 201 to vertebral members 300. In this embodiment, protectors 10 are mounted to the ends of the rods 200 to prevent the ends from causing damage and/or irritation. A protector 10 is also attached to cover one of the fasteners 201. Fastener 201 may include extending edges that could cause irritation and/or damage. Further, fastener 201 may need to be accessed during a future surgical procedure. Protector 10 prevents the growth of tissue on the fastener 201 that would make a future revision procedure more difficult. In this embodiment, protector 10 is removably attached to the fastener 201 and would be removed during the revision procedure to gain access to the fastener 201. Other embodiments of features 100 may include screws, rods, fasteners, and saddle connectors.

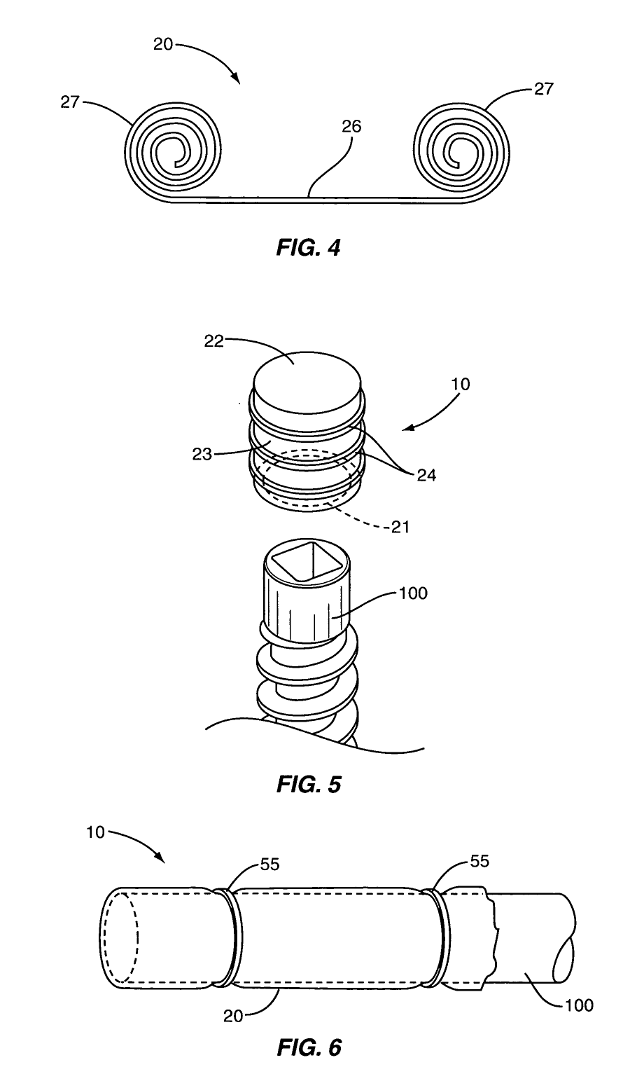

In one embodiment of the protector 10, the body 20 includes an original shape prior to attachment to the feature that corresponds to the shape of the feature 100. The original shape may closely resemble the feature 100, or vaguely resemble the feature 100. By way of example in the embodiment of FIG. 1, the body 20 includes a cap-like shape having an end 22 with a sidewall 23 that leads to an opening 21. The body 20 may substantially maintain this shape after attachment to the feature 100, or may take another shape that more closely matches the feature 100. In another embodiment of a protector 10, the body 20 includes a non-corresponding shape prior to attachment. The body 20 roughly assumes the shape of the feature 100 after attachment. FIG. 4 illustrates one embodiment of a protector 10 having an original non-conforming shape. In this embodiment, body 20 includes a rolled configuration with a first section 26 and one or more rolled ends 27. The body 20 can be positioned with the first section 26 aligned onto the feature 100 and the ends 27 can be unrolled. In one embodiment, the material is elastic and the body 20 is stretched and remains attached through tension.

Protector 10 may also be constructed of a non-elastic material that does not expand when placed on the feature 100. The non-elastic protector 100 may or may not include an original corresponding shape. One embodiment is illustrated in FIG. 5 and includes a corresponding shape prior to attachment with the fastener 100. The body 20 includes an end 22 and a sidewall 23 that form an opening 21. Opening 21 is sized to fit over the feature 100 without requiring expansion. In one embodiment, one or more ridges 24 are positioned along the sidewall 23. Ridges 24 include a different width than the opening 21 and prevent inadvertent removal of the protector 10. In one embodiment, ridges 24 include a greater width than the opening 21. In another embodiment, ridges 24 include a smaller width than the opening 21. In one embodiment, threads extend along the inner surface of the body 20 to attach with the feature 100.

Another embodiment of a protector 10 is illustrated in FIG. 6. The body 20 is constructed of a flexible, pliable material that includes an end 22 and sidewalls 23 that form an interior sized to receive the feature 100. The body 20 does not include an original corresponding shape but rather roughly conforms to the shape of the feature 100 after attachment. One or more straps 55 attach the protector 10 to the feature 100. The straps 55 may be attached to the body 20, or may be separated from the body 20 prior to attachment. In one embodiment, the straps 55 are constructed of an elastic material.

The protector 10 may remain attached to the feature 100 through a variety of different embodiments. In one embodiment as previously discussed, protector 10 is constructed of an elastic material that stretches and applies tension to maintain the position on the feature 100. In another embodiment, ridges 24 extend along an interior surface of the body 20. The ridges 24 form narrowed or enlarged interior sections within the sidewalls to catch onto the feature 100 and prevent inadvertent removal. One embodiment of ridges 24 is illustrated in FIG. 5. Another embodiment for maintaining attachment includes one or more straps 55 to attach the protector 10 to the feature 100. Straps 55 include a length to extend around the body 20 and are fixed together to hold the protector 10 onto the feature 100. FIG. 6 illustrates one embodiment with straps 55 maintaining the protector 10.

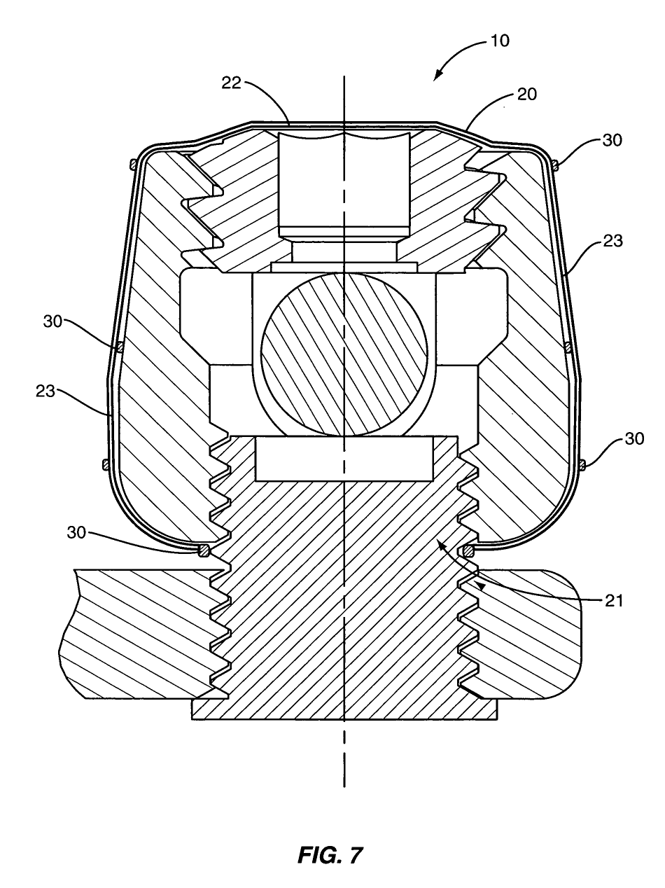

Another attachment embodiment includes one or more ribs 30 positioned along the body 20. In one embodiment, ribs 30 are constructed of an elastic material and apply a tension force to the body 20. FIG. 7 illustrates one embodiment of a protector 10 that extends around a saddle connector. Ribs 30 attached to the body 20 maintain the position of the protector 10. Ribs 30 may extend around the entirety of the body 20, or a limited distance. In one embodiment as illustrated in FIG. 7, rib 30 is placed at the opening 21 to hold the edge of the opening 21 against the feature. Additional ribs 30 are positioned along the sidewall 23 to further attach the protector 10.

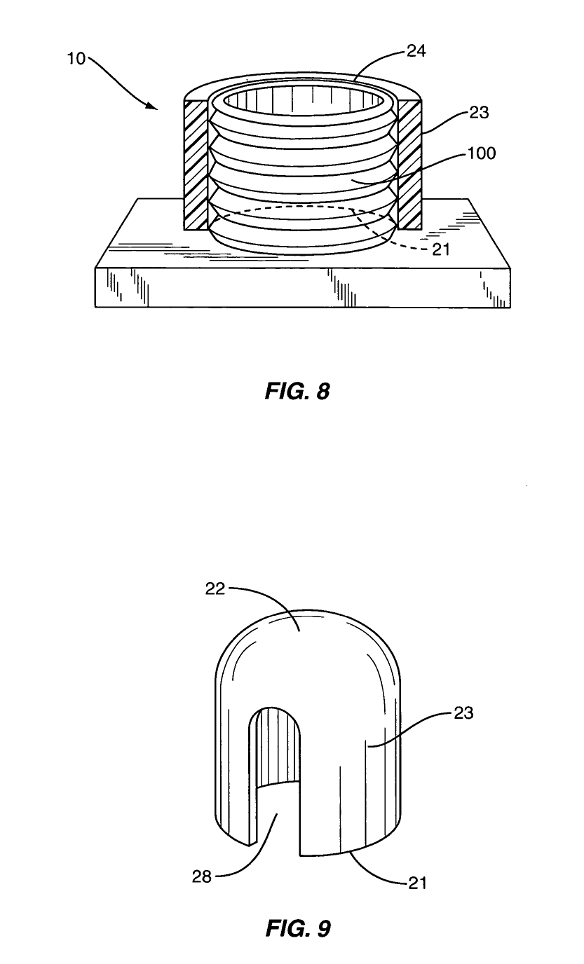

Body 20 of the protector 10 may include different shapes and sizes to extend over the feature 100. One embodiment illustrated in FIGS. 1 and 2 includes an enclosed cavity having a closed end 22 and sidewalls 23 that extend to an opening 21. In other embodiments, sidewalls 23 may include different sizes to extend differing lengths along the feature 100. FIG. 8 illustrates another embodiment having a first opening 21 for inserting the protector 10 over the feature 100. Sidewalls 23 may include a variety of lengths depending upon the context of use. A second opening 29 is positioned opposite from the first opening 21. This type of protector 10 may be used for protecting threads on the feature 100. Sidewalls 23 may include a length to extend outward beyond the top edge of the feature, or may extend only a limited length of the feature 100.

Another embodiment is illustrated in FIG. 9 and includes one or more slits 28 within the sidewall 23. In one embodiment, slits 28 are sized for the protector 10 to extend over a fastener that maintains a rod. The sidewalls 23 and end 22 substantially cover the fastener, and the rod extends outward through the slits 28. In a similar embodiment, apertures extend through the sidewalls such that the body 20 extends around the feature at a point below the rod.

The protector 10 may be attached to the feature 100 in a variety of different methods. One method includes physically grasping and attaching the protector 10. A physician grasps the protector 10 and places it onto the feature 100. The physician may further manipulate the body 20 to position it fully onto the feature 100 as necessary. Once positioned, attachment devices (e.g., straps) may be manually placed to maintain the position.

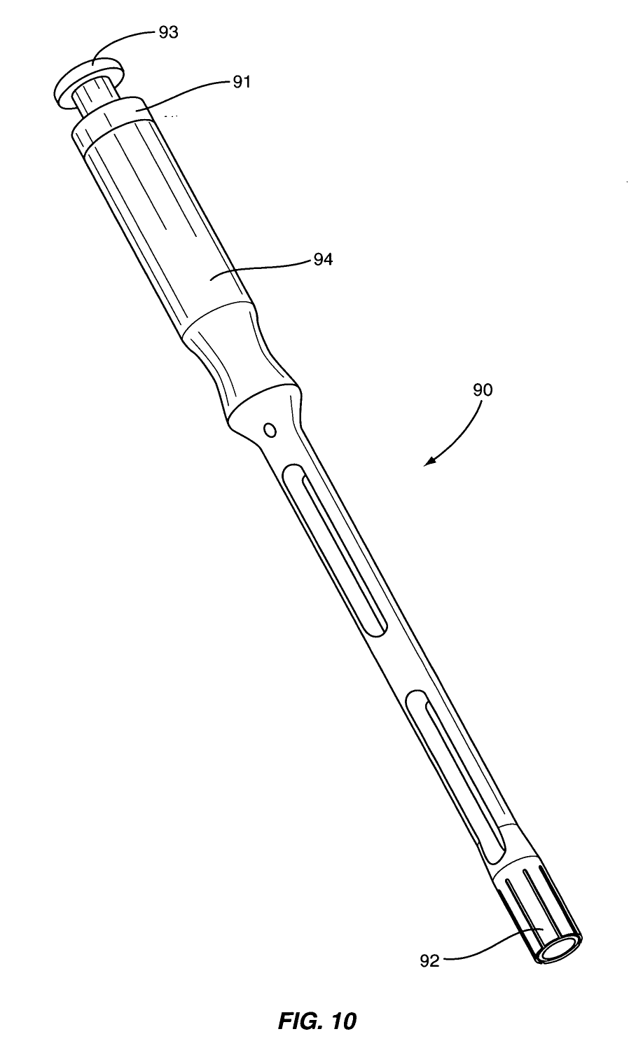

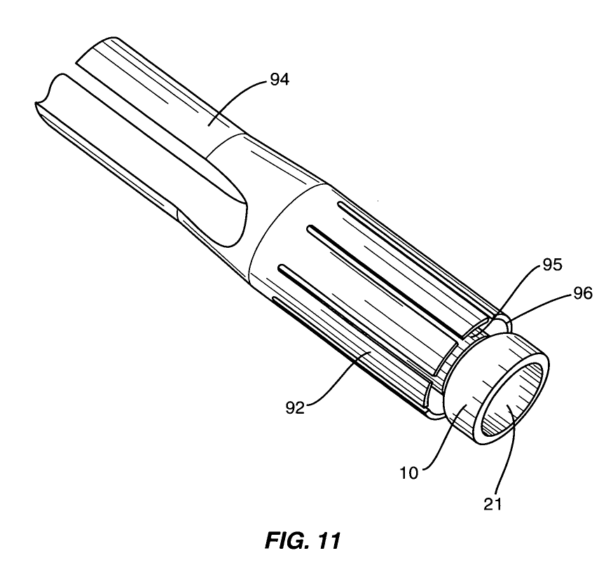

Another method includes an insertion tool 90 as illustrated in FIGS. 10 and 11. Tool 90 includes an elongated body 94 having a distal end 92 and a proximal end 91. A plunger 93 is positioned at the proximal end 91 and is operatively connected to an expulsion member 95 at the distal end 92. The distal end 92 includes a receiving section 96 formed by the body 94. A protector 10 is sized to fit within the receiving section 96 and in contact with the expulsion member 95. The tool 90 is selectively positioned between a first position with the expulsion member 95 positioned inward within the body 94 and away from the distal end 92, and a second position with the expulsion member 95 moved outward in the body 94 towards the distal end 92.

In use, the tool 90 is placed in the first position and the protector 10 is inserted into the receiving section 96 with the opening 21 facing outward. The body 94 is manipulated by the doctor and the distal end 92 is placed against the feature 100. Plunger 93 is depressed causing the expulsion member 95 to move towards the second position. This movement expels the protector 10 from the receiving section 96 and onto the feature 100. In one embodiment, body 94 includes a narrow, elongated body 94 that can be inserted into a variety of locations within the patient in a minimally invasive manner. In one embodiment, the body 94 is inverted during attachment to the feature 100. In another embodiment, the body 94 maintains the same orientation during attachment.

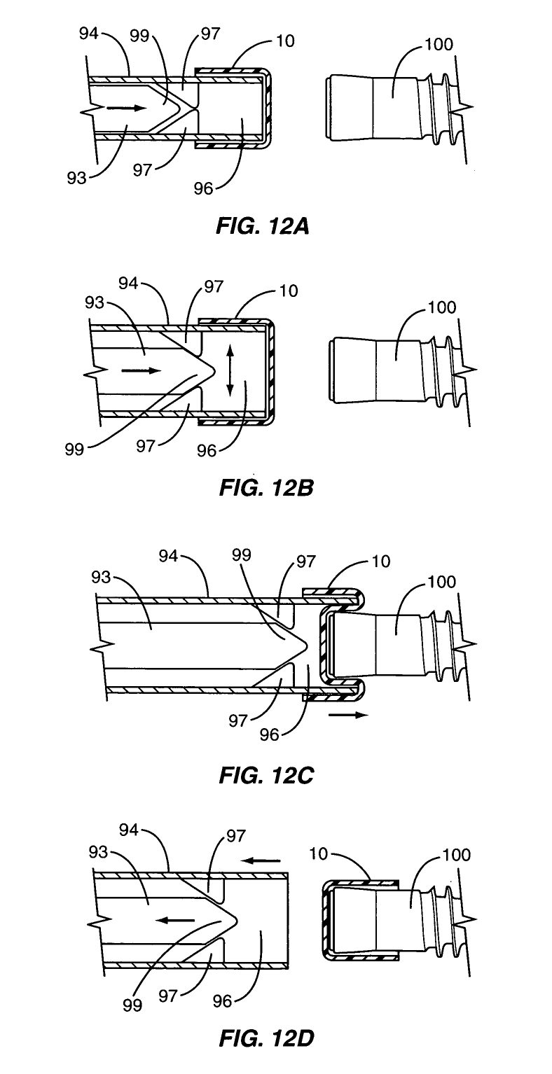

Another insertion tool 90 is similar to the embodiment of FIG. 10. This tool 90 includes a body 94 having proximal and distal ends 91, 92 and a plunger 93. As illustrated in FIGS. 12A-12D, a distal end of the plunger 93 includes an angular body 99. One or more ramps 97 are positioned on an interior sidewall of the body 94 in proximity to the distal end 92. Further, the body 94 is designed to have an expandable width. In one embodiment, the body 94 includes a circular cross sectional shape. Body 94 may also have other cross sectional shapes.

As illustrated in FIG. 12A, an elastic protector 10 is placed onto the distal end 92 over the opening 96 and may extend along a section of the body 94. The angular body 99 of the plunger 93 is placed away from the one or more ramps 97 thus causing the distal end 92 to have a first width. As illustrated in FIG. 12B, the plunger 93 is moved forward causing the angular body 99 to contact the ramps 97. This contact causes the width of the distal end 92 to increase thereby causing the width of the protector 10 to increase. The plunger 93 and connected angular body 99 are moved in a distal direction along the ramps 97 until reaching a desired width of the distal end 92 and protector 10. In one embodiment, the tip of the angular body 99 and/or the surfaces of the ramps 97 are lubricated to reduce friction and ease the expansion. In one embodiment, the lubrication includes a surface treatment or surface coating. In one embodiment, the inner surface of the protector 10 is lubricated to ease the expansion. This lubrication may also ease removal of the protector 10 from the body 94.

In one embodiment, the tool 90 is then moved forward for the distal end 92 to extend over the feature 100 as illustrated in FIG. 12C. This movement causes the protector 100 to contact the feature 100. Continued forward movement causes protector 100 to move from the body 94 to the feature 100. This movement causes the protector 100 to invert as it moves from the body 94 onto the feature 100. The outer side of the protector 100 while positioned on the body 94 becomes the inner side when positioned on the feature 100. In this embodiment, the protector 100 covers the end and sides of the feature 100. The tool 90 is then moved in a backward direction while still in the expanded state. After moving beyond the feature 100, the angular body 99 is moved in proximal direction away from the ramps 97 causing the width to reduce to the original size.

In one embodiment, the protector 10 includes an easily-identifiable color that may ease locating the protector 10 during a revision procedure. Such a color may be different than the color of the feature 100, and the color of the surrounding tissue. The protector 10 may include a single color, or may include multiple colors. In one embodiment, a first section adjacent to the opening 21 includes a first color and a second section adjacent to the end 22 includes a second, different color.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. In one embodiment, a first section of the body 20 is constructed of a first material, and a second section is constructed of a second, different material. In one embodiment, the first section is constructed of an elastic material, and a second section is constructed of a non-elastic material. The present embodiments are, therefore, to be considered in all respects as illustrative and not restrictive, and all changes coming within the meaning and equivalency range of the appended claims are intended to be embraced therein.

What is claimed is:

1. A device to protect a feature on an implant comprising a body having a closed end, a sidewall continuously extending outward from the end, and an opening formed by the sidewall and leading into a cavity formed by the end and the sidewall, the body constructed of an elastic material that is expandable from a first size with the opening smaller than the feature to a second size with the opening larger than the feature.

2. The device of claim 1, wherein the body includes a defined shape prior to attachment that corresponds to the feature.

3. The device of claim 1, further comprising an elastic rib attached to the body to apply tension and maintain the body attached to the feature.

4. The device of claim 1, further comprising elongated straps having opposing ends that connect together to attach the body to the feature.

5. The device of claim 1, wherein the body includes a non-corresponding shape prior to attachment that is different than the feature.

6. The device of claim 5, wherein the body includes a rolled configuration with at least one rolled section.

7. A device to protect a feature on an implant comprising a flexible, elastic cover that is expandable to fit onto the feature and apply a tension force to maintain attachment on the feature.

8. The device of claim 7, wherein the cover comprises a defined shape prior to attachment that corresponds to the feature.

9. The device of claim 7, wherein the cover includes a non-corresponding shape prior to attachment that is different than the feature.

10. The device of claim 7, wherein the device comprises an end and a sidewall that extends from the end and forms a cavity that receives the feature.

11. The device of claim 7, wherein the device is substantially cylindrical having a sidewall with first and second openings on opposite ends of the sidewall.

12. The device of claim 7, wherein the device includes a first color that is different than a color of the feature.

13. A device to protect a feature on an implant comprising a body having a closed end, a sidewall continuously extending outward from the end, and an opening formed by the sidewall and leading into a cavity formed by the end and the sidewall, the body constructed of a deformable material that deforms during attachment to the feature.

14. The device of claim 13, further comprising one or more ridges positioned on an inner side of the sidewall and extending into the cavity to form narrow sections within the cavity to maintain the body attached to the feature.

15. The device of claim 13, further comprising a slit positioned within the body and extending from the opening towards the end.

16. The device of claim 13, wherein the body is constructed of an elastic material.

17. A method of protecting a feature on an implant, the method comprising the steps of:
aligning a protector relative to the feature;
expanding the protector from a first size to a second size;
placing the protector onto the feature while in the second size; and
returning the protector from the second size towards the first size and applying a tension force to the feature to maintain the protector attached to the feature.

18. The method of claim 17, wherein the step of aligning the protector relative to the feature comprises positioning a closed end of the protector against a first surface of the protector.

19. The method of claim 17, further comprising changing a shape of the protector from a first shape prior to attachment to a second shape after attachment.

20. The method of claim 17, further comprising changing a shape of the protector to substantially match the feature.

21. The method of claim 17, further comprising aligning first and second openings relative to the feature and contacting a sidewall of the protector against the feature.

22. The method of claim 17, further comprising removing the protector from the feature after a period of time has expired.

23. The method of claim 17, further comprising inverting the orientation of the protector while placing the protector onto the feature.

24. A method of protecting a feature on an implant, the method comprising the steps of:
aligning a protector relative to the feature while the protector includes a first shape;
placing the protector onto the feature and deforming the protector from the first shape to a second shape that extends onto the feature; and
maintaining the protector on the feature.

25. The method of claim 24, comprising deforming the protector from the first shape that is non-corresponding to the feature to the second shape that corresponds to the feature.

26. The method of claim 24, further comprising removing the protector from the feature after a period of time.

27. The method of claim 24, further comprising inverting the protector during placement onto the feature.
